(19) United States
(12) Patent Application Publication
(10) Pub. No.: US 2009/0148872 A1
(43) Pub. Date: Jun. 11, 2009

(54) LIPOPROTEIN SENSOR

(75) Inventors: Lindy Jane Murphy, Yarnton (GB); Herbert Frank Askew, Yarnton (GB)

Correspondence Address: DARBY & DARBY P.C., P.O. BOX 770, Church Street Station, New York, NY, 10008-0770, US

(73) Assignee: OXFORD BIOSENSORS LTD., Yarnton (GB)

(21) Appl. No.: 12/282,926

(22) PCT Filed: Apr. 5, 2007
(86) PCT No.: PCT/GB07/01279

§ 371 Date: Oct. 6, 2008

(30) Foreign Application Priority Data
Apr. 6, 2006 (GB) 0606998.3

(51) Int. Cl. C12Q 1/60; C12M 1/34; C07K 14/435

(52) U.S. Cl. 435/11; 435/287.1; 530/359

(57) ABSTRACT

According to the present invention there is provided a biosensor comprising a substrate containing a biochemical analyte, an enzyme system, a low molecular weight glycol ether and a detection means. The biochemical analyte is a low density lipoprotein. The enzyme system contains a cholesterol enzyme such as cholesterol esterase, cholesterol oxidase or cholesterol dehydrogenase.

The present invention relates to the use of a glycol ether in a sensor. In particular, the present invention relates to the use of a glycol ether in a biosensor which selectively solubilises low density lipoprotein in cholesterol (LDL) with minimal interaction with high density lipoprotein in cholesterol thus enabling detection of LDL.

Cholesterol plays an important part in normal body function. It plays a part in the development of cell tissue, reproduction of cell membranes, hormones, and serves other functions. However, a high level of cholesterol in the blood increases the risk of coronary heart disease which can lead to a heart attack. In addition, it is known to be associated with an increased risk of stroke. A patient suffering high levels of blood cholesterol is considered to be suffering from hypercholesterolemia.

There are two main sources of cholesterol in the body. The first main source is from the body itself. The other main source is from foods such as meat, poultry, fish and dairy products. Foods that are high in saturated fat encourage the body to increase the production of cholesterol.

Cholesterol is transferred to and from cells by special carriers known as lipoproteins. This is because it is insoluble in blood. There are two main types of lipoprotein. These are low density lipoproteins (LDL), and high density lipoproteins (HDL). LDL is known to be a “bad” form of cholesterol carrier whereas the HDL is known to be the “good” form of cholesterol carrier. LDL cholesterol tends to build up in the inner wall of arteries resulting in plaque deposits which clogs the arteries, leading to increased risk of either a heart attack or a stroke. The desired level of LDL cholesterol in the blood is about 100 mg/dl. A higher level (greater than 160 mg/dl) presents an increased risk of heart disease.

HDL cholesterol is believed to protect the body against such an increased risk of heart disease. It is believed that HDL carries cholesterol away from the arteries and back to the liver. In addition, HDL may also remove excess cholesterol from plaque deposits already present in the arteries.

There has, therefore, been much effort in developing sensors which can differentiate between the amounts of LDL cholesterol and HDL cholesterol in the blood.

Traditionally, the amount of cholesterol in low density lipoprotein has been determined using differential ultracentrifugation. However, this requires special equipment and can take a long time to obtain the required measurements.

More recently, sensors which are easier to use and provide more reliable results have been developed. Such sensors are generally known as biosensors.

Biosensors are analytical tools combining a biochemical recognition component or sensing element with a physical transducer. They have wide application in such diverse fields as personal health monitoring, environmental screening and monitoring, bioprocess monitoring, and within the food and beverage industry.

The biological sensing element can be an enzyme, antibody, DNA sequence, or even a microorganism. The biochemical component serves to selectively catalyze a reaction or facilitate a binding event. The selectivity of the biochemical recognition event allows for the operation of biosensors in a complex sample matrix, i.e., a body fluid. The transducer converts the biochemical event into a measurable signal, thus providing the means for detecting it. Measurable events range from spectral changes, which are due to production or consumption of an enzymatic reaction's product/substrate, to mass change upon biochemical complexation. In general, transducers take many forms and they dictate the physicochemical parameter that will be measured. Thus, the transducer may be optically-based, measuring such changes as optical absorption, fluorescence, or refractive index. It may be mass-based, measuring the change in mass that accompanies a biologically derived binding reaction. Additionally, it may be thermally based (measuring the change in enthalpy (heat) or impedance based (measuring the change in electrical properties) that accompanies the analyte/bio-recognition layer interaction or electrochemistry based.

Biosensors offer the convenience and facility of distributed measurement, that is, the potential ability to take the assay to the point of concern or care. Properly designed and manufactured, biosensor devices may be conveniently mass-produced. There are, however, several limitations to the use of biosensors. These include a vulnerability of the transducer to fouling and interferences.

Enzyme based biosensors are widely used in the detection of analytes in clinical, environmental, agricultural and biotechnological applications. Analytes that can be measured in clinical assays of fluids of the human body include, for example, glucose, lactate, cholesterol, bilirubin and amino acids. Levels of these analytes in biological fluids, such as blood, are important for the diagnosis and the monitoring of diseases.

The sensors which are generally used in enzyme based systems are provided as either point of care or over the counter devices. They can be used to test fresh, unmodified, whole finger prick blood samples, to determine the concentrations of total cholesterol, triglycerides, HDL and LDL, within, for example, 1 to 2 minutes of adding the sample to a device (note this time is not fixed and could be subject to significant variations). These four parameters, in combination, have been clinically proven to provide a very good indication of the risk of heart disease in adults. It is well known that high cholesterol is asymptomatic. Thus, it is recommended that every adult should have a test to assess their risk. If their risk is found to be high it can be significantly reduced by correct management of either diet alone, or in combination with therapeutic drugs.

In one example of such an enzyme based biosensor there is utilised an electrochemical assay to detect the analyte in question. Use is made of a change in the oxidation state of a mediator that interacts with an enzyme which has reacted with the analyte to be determined. The oxidation state of the mediator is chosen so that it is solely in the state which will interact with the enzyme on addition of the substrate. The analyte reacts with the mediator via the enzyme. This causes the mediator to be oxidised or reduced (depending on the enzymatic reaction) and this change in the level of mediator can be measured by determining the electrochemical signal for example current generated at a given potential.

Conventional microelectrodes, typically with a working microelectrode and a reference electrode can be used. The working electrode is usually made of palladium, platinum, gold or carbon. The counter electrode is typically carbon, Ag/AgCl, Ag/Ag₂SO₄, palladium, gold, platinum, Cu/CuSO₄, Hg/HgO, Hg/HgCl₂, Hg/HgSO₄ or Zn/ZnSO₄.

The working electrode can be in a well of a receptacle forming said microelectrode. Examples of microelectrodes which can be used in are those disclosed in WO03/097860, which is incorporated by reference herein in its entirety.

The prior art teaches a number of methods of detecting LDL cholesterol in a sample such as blood, serum or plasma. Many of these prior art methods for determining the concentration of cholesterol are based on assessment of various properties, such as a change in colour.

EP 1 434 054, WO 03/102596 and JP 2004-354284 disclose a biosensor which uses a polyethylene glycol ether. U.S. Pat. No. 6,762,062 discloses a method for determining cholesterol in low density lipoprotein. The method is based upon measuring the total level of cholesterol in a sample and the levels of cholesterol in the non LDL fractions (HDL, VLDL and chylomicroms). The amount of LDL cholesterol can then be determined by simply subtracting one amount from the other. U.S. Pat. No. 6,342,364 and JP 2001-343348 also disclose LDL detection systems based on the use of electrochemical cells.

It would, therefore, be advantageous to have a detection system which is simple to use, but produces consistent and reliable results and does not require a change in colour as part of the detection methodology.

According to a first aspect of the present invention there is provided a biosensor comprising a substrate containing a biochemical analyte, an enzyme system, a low molecular weight glycol ether and a detection means.

Typically the substrate is a biological fluid such as blood or plasma. The biochemical analyte determined from said biological fluid can be a lipoprotein, usually a low density lipoprotein.

The enzyme system can contain a cholesterol enzyme such as cholesterol esterase, cholesterol oxidase or cholesterol dehydrogenase.

The low molecular weight glycol ether can be selected from the group having 1-4 repeating straight or branched alkylene glycol groups, usually said alkylene groups are ethylene, propylene and isomers thereof, butylene and isomers thereof, pentylene and isomers thereof, or combinations thereof. The glycol ethers can be substituted by an alkyl group, such as C₁-C₅ alkyl. The low molecular weight glycol ether can be selected from 2-methoxyethanol, tripropylene glycol methyl ether, diethylene glycol propyl ether, diethylene glycol butyl ether, diethylene glycol pentyl ether, 1-methoxy-2-propanol, dipropylene glycol butyl ether, tripropylene glycol butyl ether, glycerol ethoxylate-co-propoxylate triol, neopentyl glycol ethoxylate, propxyethanol, triethylene glycol methyl ether, propylene glycol propyl ether, 1-tert-butoxy-2-propanol, dipropylene glycol propyl ether, tripropylene glycol propyl ether or dipropylene glycol tert-butyl ether.

The biosensor can further include an aqueous buffer solution. The buffer solution typically has a pH of 5 to 10. More preferably, pH range can be 7-10.

The ionic strength or salt strength of the solution of the biosensor can be increased such that the selectivity for low density lipoprotein is improved. The ionic strength can be increased by adding a salt selected from the group consisting of potassium chloride, magnesium sulphate, ruthenium hexamine chloride, sodium chloride, calcium chloride, magnesium chloride, lanthanum chloride, sodium sulphate or magnesium sulphate.

The detection means can be in the form of an electrochemical cell.

According to a second aspect of the present invention there is provided a detection system for measuring the amount of a biochemical analyte in a sample comprising the steps of
a) providing a mixture of a solution of a low molecular weight glycol ether with an enzyme mixture;
b) adding a solution of the sample to be tested;
c) incubating the resulting mixture under conditions which result in a change to a measurable signal;
d) measuring the resulting change; and
e) ascertaining the amount of analyte or determining the differentiation between HDL and LDL in the original sample using a calibration curve.

The analyte can be a low density lipoprotein.

Typically the measurable signal is an electrochemical, colourimetric, thermal, piezo-electric or spectroscopic signal.

The biological analyte and reagent can be dried prior to use. The analyte and reagent can be freeze dried.

According to a third aspect of the present invention there is provided the use of a low molecular weight glycol ether for solubilising a biochemical analyte.

The low molecular weight glycol ether can be selected from the group having 1-4 repeating straight or branched alkylene glycol groups, usually said alkylene groups are ethylene, propylene and isomers thereof, butylene and isomers thereof, pentylene and isomers thereof, or combinations thereof. The glycol ethers can be substituted by an alkyl group, such as C₁-C₅ alkyl. The low molecular weight glycol ether can be selected from 2-methoxyethanol, tripropylene glycol methyl ether, diethylene glycol propyl ether, diethylene glycol butyl ether, diethylene glycol pentyl ether, 1-methoxy-2-propanol, dipropylene glycol butyl ether, tripropylene glycol butyl ether, glycerol ethoxylate-co-propoxylate triol, neopentyl glycol ethoxylate, propoxyethanol, triethylene glycol methyl ether, propylene glycol propyl ether, 1-tert-butoxy-2-propanol, dipropylene glycol propyl ether, tripropylene glycol propyl ether or dipropylene glycol tert-butyl ether.

The glycol ether can be used to solubilise a lipoprotein such as low density lipoprotein cholesterol.

In a fourth aspect of the invention the ionic strength of the solution assists in the differentiation obtained between the HDL and the LDL cholesterols. It has been found that a change in the ionic strength or salt concentration of the liquid influences the relative extent of the reaction to the two cholesterols. Accordingly, there is provided the use of a salt to increase the ionic strength or salt concentration of a solution containing a low density lipoprotein, a high density lipoprotein and a glycol ether wherein the increase in ionic strength of said solution modulates the relative solubilities of the low density lipoprotein and the high density lipoprotein.

The use of the salt to increase the ionic strength or salt concentration typically increases the solubility of the low density lipoprotein relative to the high density lipoprotein.

The ionic strength or the salt concentration of the solution can be controlled by the added salts and in the examples potassium chloride, magnesium sulphate or ruthenium hexamine chloride was used to modify the ionic strength or salt concentration of the solution. However, other salts for example, potassium chloride, magnesium sulphate, ruthenium hexamine chloride, sodium chloride, calcium chloride, magnesium chloride, lanthanum chloride, sodium sulphate or magnesium sulphate can be used.

When used herein, the following definitions define the stated term:

The term “glycol” refers to dihydric alcohols. The term “glycol ether” refers to monoalkyl ethers of dihydric or trihydric alcohols.

The term “alkyl” includes linear or branched, saturated aliphatic hydrocarbons. Examples of alkyl groups include methyl, ethyl, n-propyl, n-propyl, isopropyl, n-butyl, tert-butyl and the like. Unless otherwise noted, the term “alkyl” includes both alkyl and cycloalkyl groups.

A “biological fluid” is any body fluid or body fluid derivative in which the analyte can be measured, for example, blood, urine, interstitial fluid, plasma, dermal fluid, sweat and tears.

An “electrochemical sensor” is a device configured to detect the presence of or measure the concentration or amount of an analyte in a sample via electrochemical oxidation or reduction reactions.

A “redox mediator” is an electron transfer agent for carrying electrons between an analyte or an analyte-reduced or analyte-oxidized enzyme, cofactor or other redox active species and an electrode, either directly or via one or more additional electron transfer agents.

The term “reference electrode” includes both a) a reference electrode and b) a reference electrode that can also function as counter electrode (i.e. counter/reference electrodes), unless otherwise indicated.

The term “counter electrode” includes both a) a counter electrode and b) a counter electrode that can also function as a reference electrode (i.e., counter-reference electrode), unless otherwise indicated.

The term “measurable signal” means a signal which can be readily measured such as electrical current, electrode potential, fluorescence, absorption spectroscopy, luminescence, light scattering, NMR, IR, mass spectroscopy, heat change, or a piezo-electric change.

The term “biochemical analyte” includes any measurable chemical or biochemical substance that may be present in a biological fluid and also includes any of an enzyme, an antibody, a DNA sequence, or a microorganism.

Known biosensors that can be used in accordance with the present invention may consist of, for example, a strip with four reagent wells and a common reference; with each well having its own micro-band working electrode, such as a tubular micro-band electrode. The sensing component of the strip is provided by drying different, specially formulated, reagents comprising at least an enzyme and a mediator that will interact with specific analytes in the test sample in each well. Since, potentially, different reagents can be added and dried to each well it is clear that it is possible to complete multi-analyte testing using a single test sample. The number of wells is variable, thus the number of unique tests is variable, for example sensors using between 1 and 6 wells may be used.

Conventional microelectrodes, typically with a working microelectrode and a reference electrode can be used. The working electrode is usually made of palladium, platinum, gold or carbon. The counter electrode is typically carbon, Ag/AgCl, Ag/Ag₂SO₄, palladium, gold, platinum, Cu/CuSO₄, Hg/HgO, Hg/HgCl₂, Hg/HgSO₄ or Zn/ZnSO₄.

In a preferred microelectrode the working electrode is in a well of a receptacle forming said microelectrode. Examples of microelectrodes which can be used in accordance with the present invention are those disclosed in WO2003097860.

Embodiments of the present invention will now be described by way of example only with reference to the accompanying Figure in which:

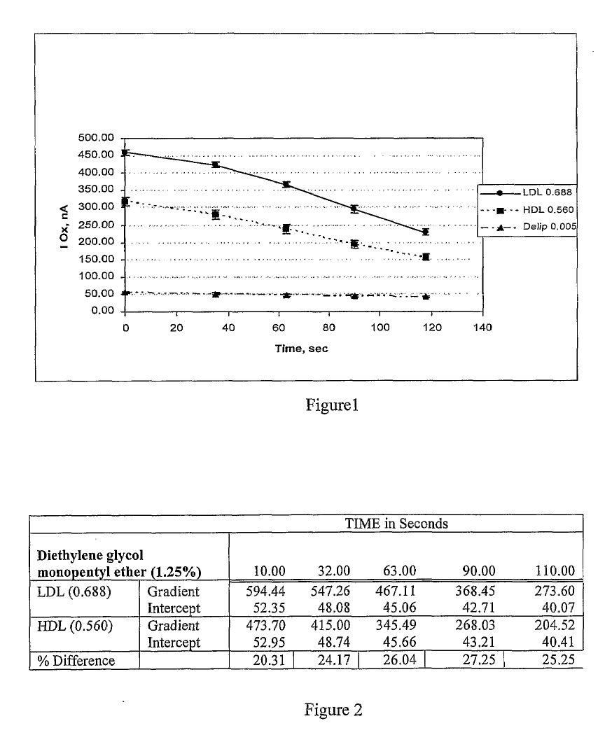

FIGS. 1 and 2 graphically illustrate the results obtained for selectively solubilising LDL over HDL when using diethylene glycol monopentyl ether (example 1).

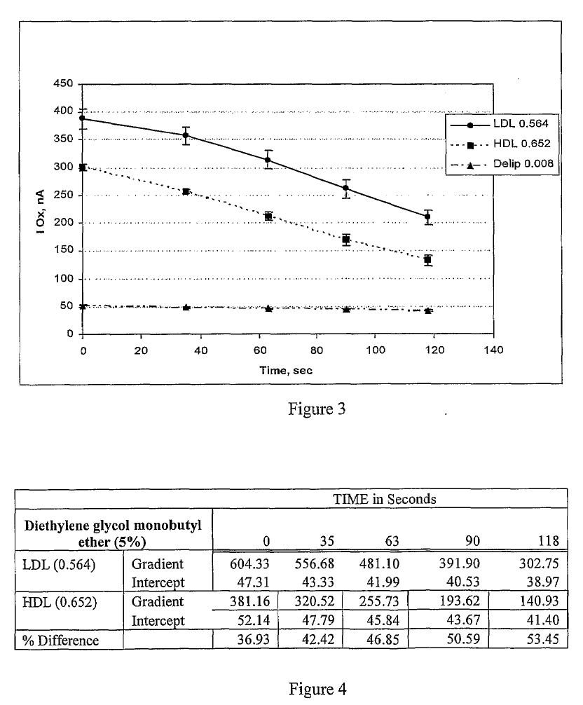

FIGS. 3 and 4 graphically illustrate of the results obtained for selectively solubilising LDL over HDL when using diethylene glycol monobutyl ether (example 1).

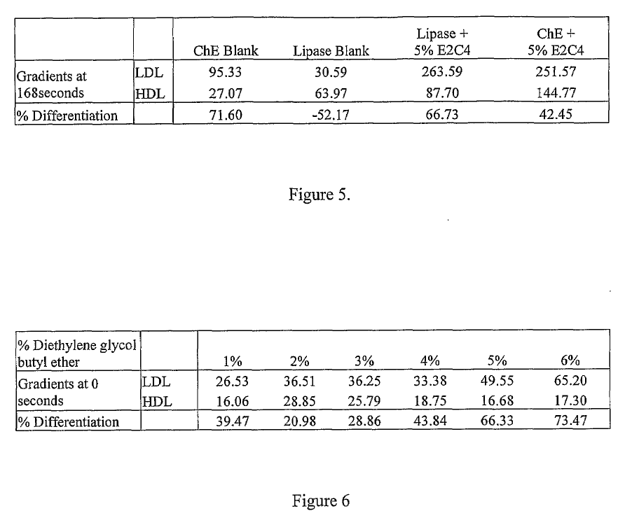

FIG. 5 shows the results from example 2. (Where E2C4 is diethylene glycol butyl ether). The gradients for each time point was used to calculate the % differentiation obtained from the measurement of LDL and HDL.

FIG. 6 shows the results from example 3. The gradients for each time point was used to calculate the % differentiation obtained from the measurement of LDL and HDL.

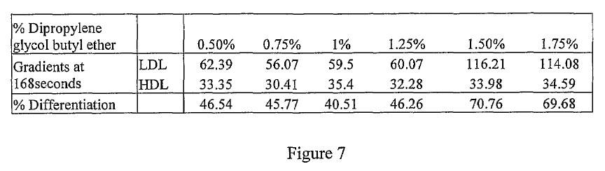

FIG. 7 shows the results from example 4. The gradients for each time point was used to calculate the % differentiation obtained from the measurement of LDL and HDL.

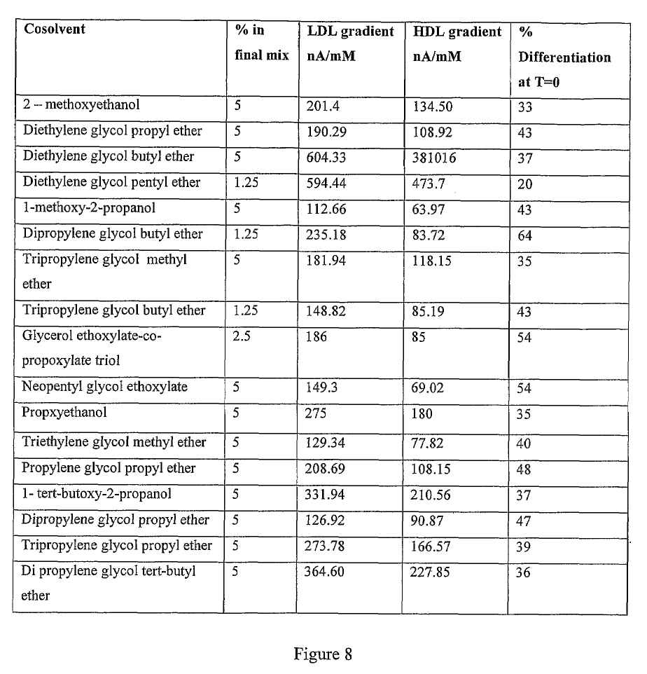

FIG. 8 shows data from example 5. The gradient at the first time point was used to calculate the % differentiation obtained between measurement of LDL and HDL.

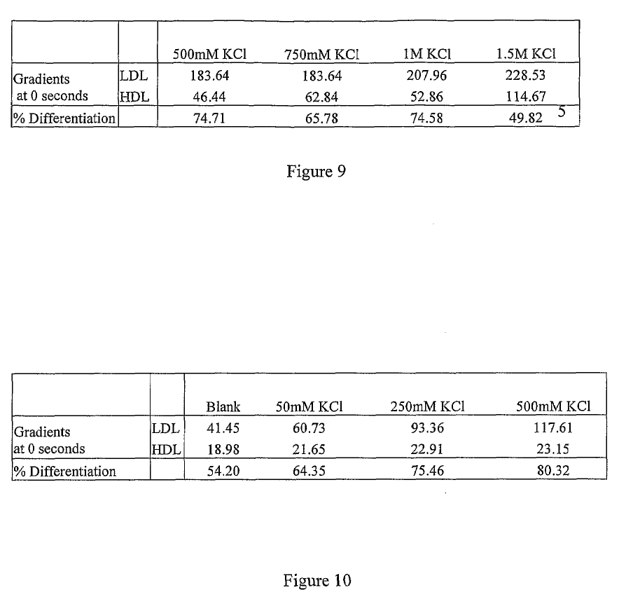

FIG. 9 shows the results from example 6. The gradients for each time point was used to calculate the % differentiation obtained between measurement of LDL and HDL.

FIG. 10 shows the data from example 7. The gradients for each time point was used to calculate the % differentiation obtained between measurement of LDL and HDL.

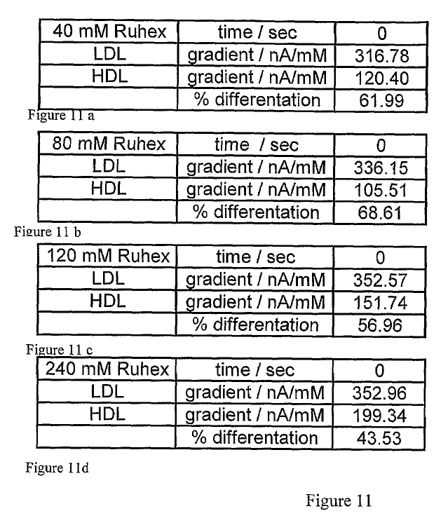

FIG. 11 a-d shows the results for the first time point 0 seconds from example 8. The gradients for each time point was used to calculate the % differentiation obtained between LDL and HDL.

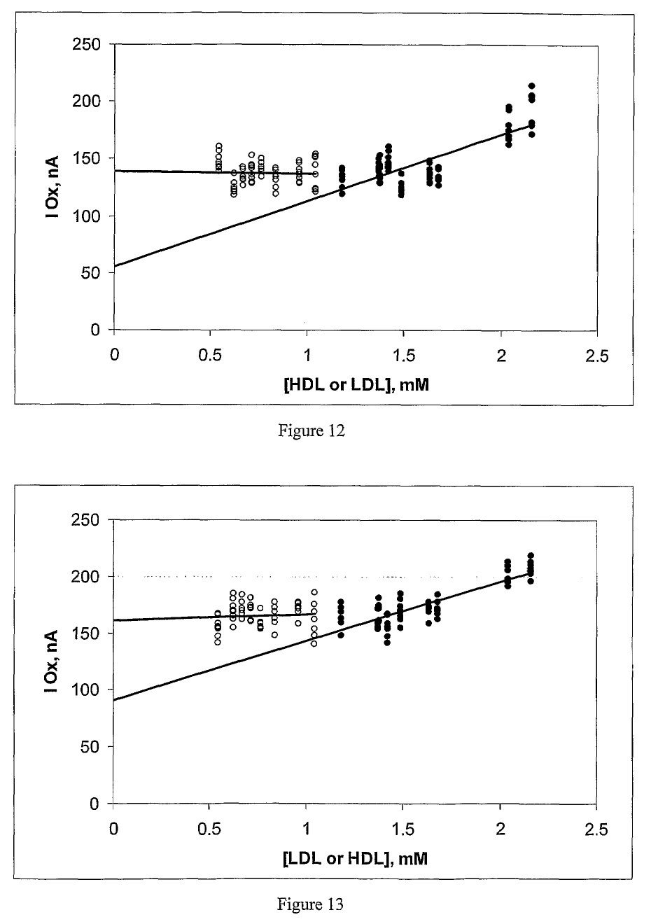

FIG. 12 shows the differentiation of plasma LDL (solid circles) and HDL (open circles) using E2C4 (example 9)

FIG. 13 shows the differentiation of plasma LDL (solid circles) and HDL (open circles) using P2C4 (example 9)

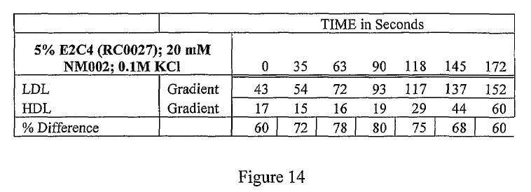

FIG. 14 shows the gradient for each time point used to calculate the % differentiation between measurement of LDL and HDL (from example 10)

### EXAMPLE 1

LDL Buffer # 1 (Tris buffer—5% glycine pH9.0)

Trizma Pre-Set Crystals, pH 9.0 (Sigma, T-1444) were dissolved in 950 mls dH₂O (dH₂O=deionised water) and the pH recorded. Following this 50 g of glycine (Sigma, G-7403) was added to the tris solution and the pH recorded. The pH was the adjusted to within 8.8-9.2 using 10M potassium hydroxide (Sigma, P-5958) and the solution made up to 1000 mls with dH₂O and the final pH recorded (pH9.1). The solution was stored at 4° C.

Glycol Ether Solutions

A double strength glycol ether solution was made using LDL buffer #1

Diethylene glycol monopentyl ether (Sigma-Aldrich, 32285)

Approx. 2.5% (0.0218 g in 872 μl LDL buffer #1)

Diethylene glycol monobutyl ether (Sigma-Aldrich, 537640)

Approx. 10% (0.0640 g in 640 μl LDL buffer #1)

Scipac LDL & HDL Samples

The LDL (Scipac, P232-8) and HDL (Scipac, P233-8) samples were made at 10× the required concentration (due to a 1:10 dilution in the final testing mixture) using delipidated serum (Scipac, S139). The samples were then analysed using a Space clinical analyser (Schiappanelli Biosystems Inc)

Enzyme Mixture

Enzyme mixture was made at double strength using LDL buffer #1
160 mM ruthenium hexaamine (III) chloride (Alfa Aesar, 10511)
17.7 mM thionicotinamide adenine dinucleotide (Oriental Yeast Co)
8.4 mg/ml putidaredoxin reductase (Biocatalysts)
6.7 mg/ml cholesterol esterase (Sorachim/Toyobo, COE-311)
44.4 mg/ml cholesterol dehydrogenase, gelatin free (Amano, CHDH-6)

Testing Protocol

9 μl of a double strength glycol solution was mixed with 9 μl of the enzyme mixture. At T=−30 seconds, 2 μl of sample (either 10× concentrated LDL or HDL, or delipidated serum) was mixed with the resulting glycol ether:enzyme mix and 9 μl of the resulting solution placed onto an electrode. At T=0 seconds the chronoamperometry test was initiated. The oxidation current is measured at 0.15 mV at 5 time points (10, 32, 63, 90 and 110 seconds), with a reduction current measured at −0.45 mV at the final time point. Each sample was tested in duplicate.

Analysis

The data were analysed along with the concentration of LDL, HDL and delipidated serum from the Space analyser. The gradients of response to HDL and LDL for each time point was used to calculate the % differentiation obtained from the measurement of LDL and HDL.

FIGS. 1 and 2 graphically illustrate the results obtained for selectively solubilising LDL over HDL when using diethylene glycol monopentyl ether

FIGS. 3 and 4 graphically illustrate of the results obtained for selectively solubilising LDL over HDL when using diethylene glycol monobutyl ether.

Conclusions

Diethylene glycol monobutyl ether (5%) showed preferential differentiation for LDL of >35%. Diethylene glycol monopentyl ether (1.25%) also showed preferential differentiation for LDL but to a lesser extent of >20%.

### EXAMPLE 2

### Genzyme Cholesterol Esterase Versus Genzyme Lipase

Solutions

RuAcAc= [Ru^{III}(acac)₂(py-3-COOH)(py-3-COO)].

30 mM Ruacac solution was made up using a buffer containing 0.1M KCl, Tris pH 9.0, 5% glycine.

Diethylene Glycol Butyl Ether Solution: A 10% glycol ether solution was made using RuAcac solution.

Enzyme mixture was made using Ruacac solution:
17.7 mM thionicotinamide adenine dinucleotide
8.4 mg/ml putidaredoxin reductase
6.7 mg/ml cholesterol esterase or 6.7 mg/ml lipase
44.4 mg/ml cholesterol dehydrogenase, gelatin free

The LDL (Scipac, P232-8) and HDL (Scipac, P233-8) samples were made using delipidated serum (Scipac, S139). The samples were then analysed using a Space clinical analyser (Schiappanelli Biosystems Inc)

Testing Protocol

9 μl of a either a double strength glycol ether solution (or Ruacac solution without glycol ether) was mixed with 9 μl of the enzyme mixture. At T=−30 seconds, 2 μl of sample (LDL or HDL, or delipidated serum) was mixed with the resulting glycol ether:enzyme mix and 9 μl of the resulting solution and placed on an electrode. The electrode is as described in WO200356319. At T=0 seconds the chronoamperometry test was initiated. The oxidation current is measured at 0.15 mV at 7 time points (0, 28, 56, 84, 112, 140 and 168 seconds), with a reduction current measured at −0.45 mV at the final time point. Each sample was tested in duplicate.

Results

Where E2C4 is diethylene glycol butyl ether.

The gradients for each time point was used to calculate the % differentiation obtained from the measurement of LDL and HDL and are shown in FIG. 5.

Conclusions

In the presence of 5% diethylene glycol butyl ether, using either cholesterol esterase or lipase confers LDL differentiation on the enzyme mix, although the differentiation to LDL is highest with cholesterol esterase.

These data indicate that the differentiation with lipase switches from HDL differentiation to LDL differentiation by the addition of diethylene glycol butyl ether.

This shows that the diethylene glycol butyl ether has a stronger effect on LDL differentiation than the type of cholesterol ester hydrolyzing enzyme.

### EXAMPLE 3

### Identifying Optimal Concentration of Diethylene Glycol Butyl Ether to Selectively Solubilise LDL

The aim of the experiment was to titrate diethylene glycol monobutyl ether to identify the optimal concentration for selectively solubilising LDL with minimal interaction with HDL for the purpose of detecting LDL.

Solutions:

RuAcAc Solution: 30 mM RuAcac made up using buffer containing Tris pH9.0, 10% Sucrose and 0.1M KCl.

Glycol ether solutions were made up at 12%, 10%, 8%, 6%, 4% and 2% diethylene glycol butyl ether in the above Ruacac solution.

The enzyme mixture (containing cholesterol esterase) and LDL and HDL samples were made up to the same recipes as in example 2.

Method:

The experiment and analysis was carried out according to the method described in experiment 2. The results are shown in FIG. 6.

Conclusions

The gradient of response to LDL increased with increasing concentration of diethylene glycol butyl ether. This resulted in the differentiation to LDL being highest at 6% diethylene glycol butyl ether.

### EXAMPLE 4

### Identifying Optimal Concentration of Dipropylene Glycol Butyl Ether to Selectively Solubilise LDL

The aim of the experiment was to vary the concentration of dipropylene glycol monobutyl ether in order to identify optimal concentration to selectively solubilise LDL with minimal interaction with HDL for the purpose of detecting LDL.

Solutions

30 mM Ruacac buffer, enzyme solution (containing cholesterol esterase) and HDL or LDL Scipac samples were prepared as described in example 2.

Glycol ether solutions were made using 3.5%, 3%, 2.5%. 2%. 1.5% and 1% dipropylene glycol butyl ether in the Ruacac solution previously described.

Methods: The experiment was carried out as described in example 2. The results are shown in FIG. 7.

Conclusions

As the concentration of dipropylene glycol butyl ether was increased, increased gradient of response to LDL was obtained. This resulted in increased differentiation to LDL. Highest differentiation was obtained at 1.5 and 1.75% dipropylene glycol butyl ether.

### EXAMPLE 5

### Identifying Agents that Show Increased Selectivity for LDL

The aim of the experiment was to identify agents that show selectivity for LDL with minimal interaction with HDL for the purpose of detecting LDL.

Solutions

Glycol Ether Solutions: each glycol ether solution was made using Tris buffer, pH 9.0, 5% glycine. The amounts below give a double strength glycol ether solution. Please note that due to small variations in weighing, the percentages are only approximations:
2-methoxy ethanol (Aldrich 185469)
10% (0.0477 g in 477 μl buffer)
Triethylene glycol methyl ether (Fluka 90450)
10% (100 μl+900 μl buffer)
Diethylene glycol propyl ether (Aldrich 537667)
10% (0.0947 g in 947 μl buffer)
Diethylene glycol butyl ether (Aldrich 537640)
10% (0.0640 g in 640 μl buffer)
Diethylene glycol pentyl ether (Fluka 32285)
2.5% (0.0218 g in 872 μl buffer)
1-methoxy-2-propanol (Aldrich 65280)
10% (0.0459 g in 459 μl buffer)
Dipropylene glycol butyl ether (Aldrich 388130)
2.5% (0.0121 g in 484 μl buffer)
Tripropylene glycol methyl ether (Aldrich 30,286-4)
10% (0.0463 g in 463 μl buffer)
Tripropylene glycol butyl ether (Aldrich 48,422-9)
2.5% (0.0176 g in 704 μl buffer)
Glycerol ethoxylate-co-propoxylate triol (Aldrich 40,918-9)
5% (0.0534 g in 1.068 ml buffer)
Neopentyl glycol ethoxylate (Aldrich 410276)
10% (0.0619 g in 619 μl buffer)
Propylene glycol propyl ether (Sigma-Aldrich 424927)
10% (0.0444 g in 444 μl buffer)
1-tert-butoxy-2-propanol (Sigma-Aldrich 433845)
10% (0.0470 g in 470 μl buffer)
Dipropylene glycol propyl ether (Sigma-Aldrich 484210)
10% (0.0458 g in 458 μl buffer)
Tripropylene glycol propyl ether (Sigma-Aldrich 469904)
10% (0.0435 g in 435 μl buffer)
Dipropylene glycol tert-butyl ether (Sigma-Aldrich 593346)
10% (0.0417 g in 417 μl buffer)
2-Propoxyethanol (Sigma-Aldrich 82400)
10% (0.0444 g in 444 μl buffer)

Scipac LDL & HDL Samples: The LDL and HDL samples were made up using delipidated serum.

Enzyme Mixture

Enzyme mixture was made up using Tris buffer, pH9.0, 5% glycine described above to contain:
160 mM ruthenium hexaammine (III) chloride
17.7 mM thionicotinamide adenine dinucleotide
8.4 mg/ml putidaredoxin reductase
6.7 mg/ml cholesterol esterase
44.4 mg/ml cholesterol dehydrogenase, gelatin free

Testing Protocol

9 μl of glycol solution was mixed with 9 μl of the enzyme mixture. At T=−30 seconds, 2 μl of sample (either LDL, HDL, or delipidated serum) was mixed with the resulting glycol ether:enzyme mix and 9 μl of the resulting solution placed onto an electrode. The electrode is as described in WO200356319. At T=0 seconds the chronoamperometry test was initiated. The oxidation current is measured at 0.15 mV at 5 time points (10, 32, 63, 90 and 110 seconds), with a reduction current measured at −0.45 mV at the final time point. Each sample was tested in duplicate.

Results

The data were analysed and the gradient at the first time point was used to calculate the % differentiation obtained between measurement of LDL and HDL. The results are shown in FIG. 8.

### EXAMPLE 6

### KCl Titration 500 to 1500 mM

The aim of the experiment was to investigate the effect of increased ionic strength on the LDL and HDL response in the presence of diethylene glycol mono butyl ether.

Solutions

30 mM Ruacac Solution: 30 mM RuAcac, Tris pH 9.0, 5% glycine, 5% diethylene glycol butyl ether

KCl solutions at 3M, 2M, 1.5M and 1M KCl were made up in the Ruacac solution described above.

Enzyme mixture was made in the Ruacac solution described above:
17.7 mM thionicotinamide adenine dinucleotide
8.4 mg/ml putidaredoxin reductase
6.7 mg/ml cholesterol esterase
44.4 mg/ml cholesterol dehydrogenase

Scipac LDL & HDL Samples were made up in delipidated serum.

Testing Protocol

9 μl of the KCl solution was mixed with 9 μl of the enzyme mixture. At T=−30 seconds, 2 μl of sample was mixed with the resulting KCl:enzyme mix and 9 μl of the resulting solution placed onto an electrode. At T=0 seconds the chronoamperometry test was initiated. The oxidation current is measured at 0.15 mV at 7 time points (0, 32, 64, 96, 128, 160 and 192 seconds), with a reduction current measured at −0.45 mV at the final time point. Each sample was tested in duplicate.

The data were analysed. The gradients for each time point was used to calculate the % differentiation obtained between measurement of LDL and HDL. The results are shown in FIG. 9.

Conclusions

Increasing the concentration of KCl to very high concentration (1.5 M) reduced the differentiation to LDL by increasing the gradient of response to HDL. High differentiation to LDL was obtained at 500, 750 and 1 M KCl.

### EXAMPLE 7

### KCl Titration 0 to 500 mM

The aim of the experiment was to investigate the effect of ionic strength on the LDL and HDL response in the presence of diethylene glycol butyl ether.

Solutions

30 mM RuAcac solution made up in buffer containing Tris pH 9.0, 5% glycine, 5% diethylene glycol butyl ether solution.

KCl solutions were made up at 1M, 500 mM, 100 mM concentrations in the Ruacac buffer described above.

Enzyme mixture was made at double strength using Ruacac solution:
17.7 mM thionicotinamide adenine dinucleotide (Oriental Yeast Co)
8.4 mg/ml putidaredoxin reductase (Biocatalysts)
6.7 mg/ml cholesterol esterase (Genzyme)
44.4 mg/ml cholesterol dehydrogenase, Gelatin free (Amano, CHDH-6)

Scipac LDL & HDL Samples were made up in delipidated serum from Scipac.

Testing Protocol

9μl of either the KCl solutions or Ruacac solution (blank) was mixed with 9 μl of the enzyme mixture. At T=−30 seconds, 2 μl of sample (either 10× concentrated LDL or HDL, or delipidated serum) was mixed with the resulting KCl:enzyme mix and 9 μl of the resulting solution placed onto an electrode. The electrode is as described in WO200356319. At T=0 seconds the chronoamperometry test was initiated. The oxidation current is measured at 0.15 mV at 7 time points (0, 32, 64, 96, 128, 160 and 192 seconds), with a reduction current measured at −0.45 mV at the final time point. Each sample was tested in duplicate.

Results

The data were analysed. The gradients for each time point was used to calculate the % differentiation obtained between measurement of LDL and HDL. The results are shown in FIG. 10.

Conclusions

Increasing the concentration of KCl in the range 0-500 mM resulted in higher differentiation to LDL with increasing concentration of KCl, due to increased gradient of response to LDL.

### EXAMPLE 8

### Investigating Ionic Strength on the Selective Solubilisation of LDL

The aim of the experiment was to investigate the effect of ionic strength on the selective solubilisation of LDL with minimal interaction with HDL for the purpose of detecting LDL, by varying the concentration of Ru hexaamine chloride mediator.

Solutions

A glycol ether solution containing 12% diethylene glycol monobutyl ether was made in Tris buffer (pH9.0, 5% glycine)

Scipac LDL & HDL Samples were made up to various concentrations using Scipac delipidated serum

Enzyme mixtures were made at double strength using TRIS buffer pH9.0, 5% glycine. Four separate enzyme mixes were prepared, containing either 80, 160, 240 or 480 mM ruthenium hexaamine chloride:
80, 160, 240 or 480 mM ruthenium hexaammine (III) chloride
17.7 mM thionicotinamide adenine dinucleotide
8.4 mg/ml putidaredoxin reductase
6.7 mg/ml cholesterol esterase
44.4 mg/ml cholesterol dehydrogenase, gelatin free

Testing Protocol

9 μl of a double strength glycol ether solution was mixed with 9 μl of the enzyme mixture. At T=−30 seconds, 2 μl of sample (either 10× concentrated LDL or HDL, or delipidated serum) was mixed with the resulting glycol ether:enzyme mix and 9 μl of the resulting solution placed onto an electrode (the electrode is as described in WO200356319). At T=0 seconds the chronoamperometry test was initiated. The oxidation current is measured at 0.15 mV at 5 time points (0, 28, 56, 84 and 112 seconds), with a reduction current measured at −0.45 mV at the final time point. Each sample was tested in duplicate.

Analysis

The data were analysed and the gradients for each time point was used to calculate the % differentiation obtained between LDL and HDL. The results for the first time point 0 seconds, are shown in tables FIGS. 11 a-d.

Conclusions

Highest differentiation to LDL was obtained with 80 mM Ru hexaamine chloride.

Whilst not wishing to be bound by any particular theory it may be supposed that the change in levels of ions present alters the relative solvating power of the co-solvent for the cholesterols until the ionic strength or the ion concentration reaches a level at which solubility becomes limited.

### EXAMPLE 9

### Plasma Calibrations with Diethylene Glycol Butyl Ether or Dipropylene Glycol Butyl Ether

The aim of the experiment was to investigate the response to plasma LDL and HDL response in the presence of diethylene glycol mono butyl ether (E2C4) or dipropylene glycol mono butyl ether (P2C4).

Solutions

KCl Buffer: Tris buffer pH 9.0, 5% glycine, 0.2M KCl

40 mM Ruacac made up using KCl buffer solution described above.

3M KCl solution was made up in the Ru acac solution described above.

Enzyme Mixtures: Enzyme mixture (without cosolvent) was made using Ruacac solution:
17.7 mM thionicotinamide adenine dinucleotide
8.4 mg/ml putidaredoxin reductase
6.7 mg/ml cholesterol esterase
44.4 mg/ml cholesterol dehydrogenase, gelatin free

Enzyme mix containing 12% E2C4: 0.0304 g E2C4 (Sigma-Aldrich) was dissolved in 253 μL enzyme mix.

Enzyme mix containing 3.5% P2C4: 0.0075 g P2C4 (Sigma-Aldrich) was dissolved in 250 μL enzyme mix.

Plasma Samples: Frozen plasma samples were defrosted for at least 30 minutes, before centrifugation for 5 minutes. The samples were then analysed using a Space clinical analyser (Schiappanelli Biosystems Inc).

Testing Protocol

For the enzyme mix containing E2C4, 1.5 μl of the 3M KCl solution was mixed with 7.5 μl of the enzyme mixture. At T=−30 seconds, 9 μl of sample (either plasma or delipidated serum) was mixed with the resulting KCl:enzyme mix and 9 μl of the resulting solution placed onto an electrode. At T=0 seconds the chronoamperometry test was initiated. The oxidation current is measured at 0.15 mV at 7 time points (0, 32, 64, 96, 128, 160 and 192 seconds), with a reduction current measured at −0.45 mV at the final time point. Each sample was tested in duplicate.

For the enzyme mix containing P2C4, at T=−30 seconds, 9 μl of the enzyme mixture was mixed with 9 μl of sample (either plasma or delipidated serum). 9 μl of the resulting solution placed onto an electrode and at T=0 seconds the chronoamperometry test was initiated as above for E2C4.

Analysis

The data were analysed. The gradients for each time point was used to calculate the % differentiation obtained between measurement of LDL and HDL.

Results

Using E2C4, the differentiation to plasma LDL was 103% at time t=0 sec (FIG. 12—HDL shown by open circles, LDL shown closed circles). Using P2C4, the differentiation to plasma LDL was 91% at t=96 sec (FIG. 13—HDL shown by open circles, LDL shown closed circles).

Conclusions

High differentiation to plasma LDL was obtained with either E2C4 or P2C4.

### EXAMPLE 10

### Experiment to Identify Agents that will Selectively Solubilise LDL with Minimal Interaction with HDL for the Purpose of Detecting LDL

Solutions

0.1M KCl Buffer=Tris buffer, pH 9.0, 5% glycine, 0.1M KCl

Glycol Ether Solutions

A double strength glycol ether solution was made using 0.1M KCl buffer:
Diethylene glycol butyl ether (Aldrich 537640)
10% (0.0958 g in 958 μl KCl buffer)

Enzyme Mixture:

Enzyme mixture was made using 0.1M KCl buffer and contained:
40 mM RuAcac
17.7 mM thionicotinamide adenine dinucleotide
8.4 mg/ml putidaredoxin reductase
6.7 mg/ml cholesterol esterase
44.4 mg/ml cholesterol dehydrogenase, gelatin free

Scipac LDL & HDL Samples:

The LDL (Scipac, P232-8) and HDL (Scipac, P233-8) samples were made at 10× the required concentration using delipidated serum (Scipac, S139). The samples were then analysed using a Space clinical analyser (Schiappanelli Biosystems Inc)

Testing Protocol

9 μl of glycol ether solution was mixed with 9 μl of the enzyme mixture. At T=−30 seconds, 2 μl of sample (LDL or HDL, or delipidated serum) was mixed with the resulting glycol ether:enzyme mix and 9 μl of the resulting solution placed onto an electrode (the electrode is as described in WO200356319). At T=0 seconds the chronoamperometry test was initiated. The oxidation current is measured at 0.15 mV at 5 time points (0, 35, 63, 90, 118, 145 and 172 seconds), with a reduction current measured at −0.45 mV at the final time point. Each sample was tested in duplicate.

Analysis

These data were analysed along with the concentration of LDL, HDL and delipidated serum from the space analyser. The gradients for each time point was used to calculate the % differentiation obtained between measurement of LDL and HDL. The results are shown in FIG. 14.

Conclusions

High differentiation to LDL was obtained with diethylene glycol butyl ether.

1. A biosensor comprising: a substrate containing a biochemical analyte; an enzyme system; a low molecular weight glycol ether; and a detection unit.

2. A biosensor as claimed in claim 1 wherein the substrate is a biological fluid.

3. A biosensor as claimed in claim 2 wherein the biochemical analyte contained in said biological fluid is a lipoprotein.

4. A biosensor as claimed in claim 3 wherein said lipoprotein is a low density lipoprotein.

5. A biosensor as claimed in claim 1 wherein the enzyme system contains a cholesterol enzyme.

6. A biosensor as claimed in claim 1 wherein the low molecular weight glycol ether is selected from the group consisting of glycol ethers having 1-4 repeating straight or branched alkylene groups.

7. A biosensor as claimed in claim 6 wherein the alkylene group is selected from the group consisting of ethylene, propylene and isomers thereof, butylene and isomers thereof, pentylene and isomers thereof, and combinations thereof.

8. A biosensor as claimed in claim 1 wherein the glycol ether is substituted by an alkyl group optionally substituted by one or more alkoxy groups.

9. A biosensor as claimed in claim 8 wherein said alkyl group is a C₁-C₅ alkyl.

10. A biosensor as claimed in claim 6 wherein said alkylene group is substituted with 1-4 alkoxy groups.

11. A biosensor as claimed in claim 10 wherein said 1-4 alkoxy groups is 1-4 ethoxy groups.

12. A biosensor as claimed in claim 1 wherein the low molecular weight glycol ether is 2-methoxyethanol, tripropylene glycol methyl ether, diethylene glycol propyl ether, diethylene glycol butyl ether, diethylene glycol pentyl ether, 1-methoxy-2-propanol, dipropylene glycol butyl ether, tripropylene glycol butyl ether, glycerol ethoxylate-co-propoxylate triol, neopentyl glycol ethoxylate, propxyethanol, triethylene glycol methyl ether, propylene glycol propyl ether, 1-tert-butoxy-2-propanol, dipropylene glycol propyl ether, tripropylene glycol propyl ether or dipropylene glycol tert-butyl ether.

13. A biosensor as claimed in claim 1, further comprising an aqueous buffer solution.

14. A biosensor as claimed in claim 13 wherein the buffer solution has an alkaline pH.

15. A biosensor as claimed in claim 13 wherein the ionic strength of the solution is increased such that selectivity for low density lipoprotein is improved.

16. A biosensor as claimed in claim 15 wherein the ionic strength of the solution is increased by adding a salt selected from the group consisting of potassium chloride, magnesium sulphate, ruthenium hexamine chloride, sodium chloride, calcium chloride, magnesium chloride, lanthanum chloride, sodium sulphate and magnesium sulphate.

17. A biosensor as claimed in claim 1 wherein the detection unit is in the form of an electrochemical cell.

18. A method for measuring the amount of a biochemical analyte in a sample, comprising the steps of:
providing a mixture of a solution of a low molecular weight glycol ether with an enzyme mixture;
adding a solution of the sample to be tested to the mixture;
incubating the resulting mixture under conditions that result in a change to a measurable signal;
measuring the resulting change; and
ascertaining the amount of analyte or determining the differentiation between HDL and LDL in the original sample using a calibration curve.

19. A method as claimed in claim 18 wherein the analyte is a low density lipoprotein.

20. A method as claimed in claim 18 wherein the measurable signal is an electrochemical, colourimetric, thermal, piezo-electric or spectroscopic signal.

21. A method as claimed in claim 18 wherein the low molecular weight glycol ether is selected from the group consisting of glycol ethers having 1-4 repeating straight or branched alkylene groups.

22. A method as claimed in claim 18, further comprising the step of drying the biological analyte and reagents prior to use.

23.-25. (canceled)

26. A method for modulating relative solubilities of low density lipoprotein and high density lipoprotein in a solution containing a low density lipoprotein, a high density lipoprotein and a glycol ether, the method comprising the step of increasing the ionic strength of said solution by using a salt.

27. The method as claimed in claim 26 wherein the increase in ionic strength increases the solubility of the low density lipoprotein relative to the high density lipoprotein.

28. The method as claimed in claim 26 wherein the salt is selected from the group consisting of potassium chloride, magnesium sulphate, ruthenium hexamine chloride, sodium chloride, calcium chloride, magnesium chloride, lanthanum chloride, sodium sulphate and magnesium sulphate.

29. The method as claimed in claim 26 wherein the concentration of said salt is in the range of 0.1M-1M.

30. The method as claimed in claim 26 wherein the ionic strength of the solution is in the range of 0.5M-1.5M.

31. A biosensor as claimed in claim 8 wherein said alkyl group is substituted with 1-4 alkoxy groups.

32. A biosensor as claimed in claim 31 wherein said 1-4 alkoxy groups is 1-4 ethoxy groups.

33. A method as claimed in claim 18 wherein the low molecular weight glycol ether is substituted by an alkyl group optionally substituted by one or more alkoxy groups.

34. A method as claimed in claim 18 wherein the low molecular weight glycol ether is 2-methoxyethanol, tripropylene glycol methyl ether, diethylene glycol propyl ether, diethylene glycol butyl ether, diethylene glycol pentyl ether, 1-methoxy-2-propanol, dipropylene glycol butyl ether, tripropylene glycol butyl ether, glycerol ethoxylate-co-propoxylate triol, neopentyl glycol ethoxylate, propxyethanol, triethylene glycol methyl ether, propylene glycol propyl ether, 1-tert-butoxy-2-propanol, dipropylene glycol propyl ether, tripropylene glycol propyl ether or dipropylene glycol tert-butyl ether.
